(19) United States
(12) Patent Application Publication
(10) Pub. No.: US 2024/0273677 A1
(43) Pub. Date: Aug. 15, 2024

(54) HYPERSPECTRAL LEARNING FOR INSTANTANEOUS SPATIOSPECTRAL IMAGING OF HEMODYNAMICS

(71) Applicant: Purdue Research Foundation, West Lafayette, IN (US)

(72) Inventors: Young L. KIM, West Lafayette, IN (US); Yuhyun JI, West Lafayette, IN (US); Sang Mok PARK, West Lafayette, IN (US); Semin KWEON, West Lafayette, IN (US); Jungwoo LEEM, West Lafayette, IN (US)

(73) Assignee: Purdue Research Foundation, West Lafayette, IN (US)

(21) Appl. No.: 18/423,669

(22) Filed: Jan. 26, 2024

Related U.S. Application Data
(60) Provisional application No. 63/444,522, filed on Feb. 9, 2023.

(51) Int. Cl. G06T 5/50; G01N 21/31; G01N 33/483; G06T 7/00; H04N 23/10

(52) U.S. Cl. CPC G06T 5/50; G01N 21/31; G01N 33/4833; G06T 7/0012; H04N 23/10; G06T 2207/10016; G06T 2207/10024; G06T 2207/10056; G06T 2207/20081; G06T 2207/20084; G06T 2207/30024

(57) ABSTRACT

A method of generating an image or video of a field of interest of a sample which includes obtaining i) a first RGB image from a field of interest of a sample, and ii) hyperspectral data from a subarea of the field of interest, extracting an RGB image of the subarea from the first RGB image of the field of interest, applying the hyperspectral data of the subarea to conduct a spectroscopic analysis of a sample thereby generating spectral parameters, inputting i) the spectral parameters, and ii) the first RGB image, collectively as training input data to a deep learning model (DLM), training the DLM with the training input data thus generating a trained DLM, obtaining and inputting a second RGB image of about the field of interest to the trained DLM, and outputting from the trained DLM a spectral map for the field of interest.

### CROSS-REFERENCE TO RELATED APPLICATIONS

The present non-provisional patent application is related to and claims the priority benefit of U.S. Provisional Patent Application Ser. 63/444,522, filed Feb. 9, 2023, the contents of which are hereby incorporated by reference in its entirety into the present disclosure.

### STATEMENT REGARDING GOVERNMENT FUNDING

This invention was made with government support under contract number TW012486 awarded by the National Institutes of Health. The government has certain rights in the invention.

### TECHNICAL FIELD

The present disclosure generally relates to systems and methods related to imaging and in particular with regards to spatiospectral imaging of hemodynamics.

### BACKGROUND

This section introduces aspects that may help facilitate a better understanding of the disclosure. Accordingly, these statements are to be read in this light and are not to be understood as admissions about what is or is not prior art.

Hyperspectral (with a high spectral resolution of ˜10 nm) or multispectral (with several spectral bands of ˜50 nm) imaging systems acquire a hyperspectral image dataset (hypercube)—a three-dimensional dataset of spectral intensity in spatial coordinates. Both spatial and spectral data are processed. Hyperspectral imaging technologies offer extensive physical and biological information in stationary or dynamic samples, ranging from microscopic settings to airborne remote-sensing environments, for a variety of applications in geology, mineralogy, agriculture, environmental science, astronomy, forensic medicine, defense, security, and biomedicine. Notably, hyperspectral imaging technologies have been reinvigorated through recent advances in data-driven machine learning. For example, deep-learning approaches have enabled the effective processing of extremely large hypercube data for classical imaging tasks and allowed for the optimization of hypercube acquisition to achieve specific tasks and objectives. Data fusion of complementary images with high-spectral or high-spatial resolutions and neural networks of improving spatial resolutions can overcome the intrinsic trade-off between spatial and spectral resolutions. However, conventional hyperspectral imaging systems still face the intrinsic limitations: bulky instruments, slow data acquisition rates, low detection efficacy (i.e., low signal-to-noise ratio), and motion artifacts.

Typically, hyperspectral imaging systems rely on mechanical scanning elements either in the spectral or spatial domains. In particular, spectral scanning systems employ a number of narrow bandpass spectral filters or dispersive optical components, whereas point scanning and line-scanning systems rely on mechanical translational components that require high precision. Thus, these scanning elements result in bulky instruments and yield suboptimal temporal resolutions. In particular, prolonged time of data acquisition time fundamentally limits dynamic imaging with a high temporal resolution. In this respect, the development of snapshot imaging technologies capable of acquiring a hypercube in a single shot manner has been an active area of research. The most common configuration used for snapshot imaging involves capturing multiple images with different spectral bands using a large-area image sensor. Specifically, large-area image sensor-based snapshot imaging is beneficial for reducing the acquisition time. Other snapshot-imaging technologies employ dispersion patterns or coded apertures projecting irradiance mixed with spatial and spectral information to further enhance the light-collection efficiency and readout rate. Subsequently, the modulated projection comprising spatial and spectral information is reconstructed into a hypercube by utilizing computational algorithms such as compressed (or compressive) sensing, or Fourier transformation.

However, previously developed hyperspectral imaging technologies with a snapshot ability face several limitations. First, typical snapshot systems are limited by the intrinsic tradeoff that must be made between the spectral and spatial resolutions; that is, an improvement in spatial resolution causes a deterioration in the number of spectral bands, thereby compromising the spectral resolution or the spatial resolution (or imaging area). Second, snapshot imaging systems are sensitive to light conditions and imaging configurations, thereby introducing significant errors in field applications. Third, the hyperspectral filter arrays, dispersion patterns, and coded apertures require high-precision fabrication or nanofabrication, including precision alignment of array components, optimized miniaturization, integration with pixel-level filters, and customized calibrations, all of which inhibit manufacturability. Consequently, the previous studies have generally been performed under laboratory settings or with stationary biological samples, thereby hampering the practical and widespread utilization.

Therefore, there is an unmet need for a novel approach in instantaneous hyperspectral imaging that enables the recovery of spectral information from conventional equipment which can provide a full reflectance spectrum in the visible range.

### SUMMARY

A method of generating an image or video of a field of interest of a sample is disclosed. The method includes obtaining i) a first Red-Green-Blue (RGB) image from a field of interest of a sample, and ii) hyperspectral data from a subarea of the field of interest. The method further includes extracting an RGB image of the subarea from the first RGB image of the field of interest, and applying the hyperspectral data of the subarea to conduct a spectroscopic analysis of a sample, thereby generating spectral parameters. The method also includes inputting i) the spectral parameters, and ii) the first RGB image, collectively as training input data to a deep learning model, and training the deep learning model with the training input data thus generating a trained deep learning model. Additionally, the method includes obtaining a second RGB image about the field of interest including areas outside of the subarea; inputting the second RGB image of the field of interest to the trained deep learning model, and outputting from the trained deep learning model a spectral map for the field of interest.

### BRIEF DESCRIPTION OF FIGURES

The patent or application file contains at least one drawing executed in color. Copies of this patent or patent application publication with color drawing(s) will be provided by the Office upon request and payment of the necessary fee.

FIG. 1 is a schematic of an experimental setup according to the present disclosure including a trichromatic Red-Green-Blue (RGB) camera (e.g., a smartphone camera) and a spectrograph camera.

FIG. 2 is a block diagram that outlines the steps of the approach provided in the present disclosure.

FIG. 3 is a schematic representing a conceptual illustration of a deep neural network that receives RGB values and returns key hemodynamic parameters (e.g., Hb and HbO₂).

FIGS. 4A and 4B are i) an architecture diagram (FIG. 4A), according to the present disclosure, and ii) key hyperparameters (FIG. 4B) of the deep neural network that directly return the hemodynamic parameters of Hb and HbO₂ from RGB input values.

FIG. 5 is a schematic representing the first hidden layer of a deep neural network which is fully connected to 18 nodes (or neurons).

FIG. 6A is a graph of spectral intensity differences vs. wavelength representing spectral intensity differences in hyperspectral data measured from a tissue phantom with oxygenated and deoxygenated hemoglobin.

FIG. 6B is a graph of spectral intensity differences vs. wavelength representing difference in the computed output values of the first hidden layer between two different RGB values of HbO₂ and Hb from the same tissue phantom in FIG. 6A.

FIGS. 7A, 7B, 7C, and 7D are images of vascular tissue in a petri dish as an experimental vascular developmental model, wherein RGB images generated by the conventional pushbroom-type hyperspectral imaging system of the prior art are shown in FIGS. 7A and 7B, a ground truth for the hemodynamic map of Hb, HbO₂, and sPO₂ corresponding to a white leghorn chicken (Gallus domesticus, Hy-Line W-36) embryo on day 8 based on FIG. 7B (shown in FIG. 7C), and the deep learning-based hemodynamic maps are shown in FIG. 7D.

FIG. 8A is a photograph of a healthy adult volunteer taking a picture with a smartphone while the inner eyelid is pulled down including an RGB image of the inner eyelid with high spatial resolution showing the field-of-view.

FIG. 8B are the peripheral hemodynamic maps of Hb, HbO₂, and sPO₂ obtained during the resting state of a healthy adult volunteer.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

In the present disclosure, the term “about” can allow for a degree of variability in a value or range, for example, within 10%, within 5%, or within 1% of a stated value or of a stated limit of a range.

In the present disclosure, the term “substantially” can allow for a degree of variability in a value or range, for example, within 90%, within 95%, or within 99% of a stated value or of a stated limit of a range.

A novel approach in instantaneous hyperspectral imaging is disclosed herein that enables the recovery of spectral information from conventional equipment which can provide a full reflectance spectrum in the visible range. Towards this end, a deep learning approach is disclosed herein which enables the recovery of spectral information from Red-Green-Blue (RGB) values acquired by a conventional trichromatic camera in order to generate a full reflectance spectrum in the visible range via computational reconstruction from an RGB image. Owing to its hardware simplicity, the disclosed novel approach can be performed by using a smartphone camera without the need for complex equipment add-ons such as dispersive optical components, e.g., spectrometers and bulky optical filters.

The disclosed novel approach includes a learning-based spatiospectral imaging method offering high spectral and temporal resolutions. The disclosed spectral learning involves mapping from a sparse spectral space (i.e., RGB values) to a dense spectral space. Specifically, the spectral resolution is in a range of 0.5-1 nm, comparable to those of scientific spectrometers and spectrographs for biomedical or biochemical applications (thereby referred to as hyperspectral learning, compared with spectral learning). First, we construct a customized dual-channel imaging setup coupled with a trichromatic camera (e.g., smartphone camera) and a spectrograph to acquire an RGB image and subarea hyperspectral data. Second, we establish a simple statistical assumption to infer the entire field-of-view from a sampled subarea and recover a hypercube from incomplete measurements. Third, we establish a machine-learning frameworks based on deep learning, incorporating the domain knowledge of tissue optics into learning algorithms. Finally, we demonstrate reliable extractions of hemodynamic parameters from several different samples of tissue phantoms, chick embryos, and human conjunctiva; the results are validated through conventional hyperspectral imaging and functional near-infrared spectroscopy. Moreover, this hyperspectral learning method is applied to smartphone video recording to demonstrate the dynamic imaging of peripheral microcirculation and ultrafast imaging of oxygen depletion in tissue phantoms.

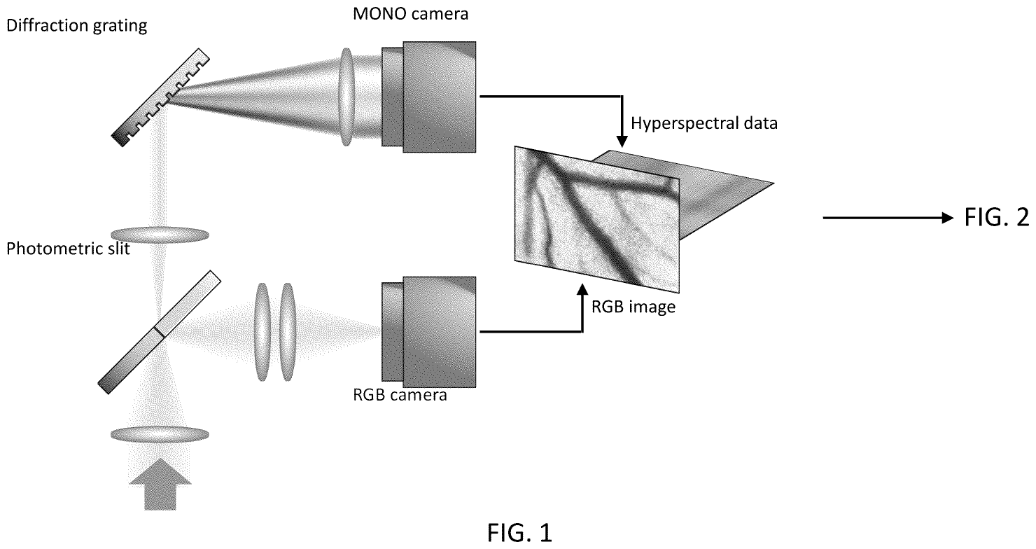

Referring to FIG. 1, an experimental setup is shown including a trichromatic RGB camera (e.g., a smartphone camera) and a spectrograph camera. Light from a sample is passed through a lens and shone upon a photometric slit which acts both as a mirror allowing visibility to the entire sample area via one or more lenses that focus light onto the RGB camera, thus allowing the RGB camera to obtain an RGB image of the entire sample area. Additionally, the photometric slit provides a slit through which light propagates onto a lens and which is shone onto a diffraction grating causing light to be dispersed into a plurality of bandwidths (i.e., different colors). The diffracted light then passes through a lens and onto the spectrograph camera which can obtain hyperspectral data from a limited portion of the sample area dictated by the slit. The position of the slit determines which limited portion of the sample area is captured by the spectrograph camera. In FIG. 1, the slit is positioned such that the hyperspectral data is of a plane that is situated about the center of the sample viewing area as captured by the RGB camera.

FIG. 1, thus illustrates the concept of hyperspectral learning for instantaneous spatiospectral imaging by significantly minimizing the number of necessary hyperspectral measurements. If hyperspectral data in a small yet representative subarea are available, a hyperspectral learning algorithm can be trained using the RGB and hyperspectral data in the subarea. This hyperspectral learning algorithm trained by the sampled (RGB and hyperspectral) data is applied to the entire image area, generating a hypercube without the need for a complete spectral or spatial scan. Thus, the key advantages of hyperspectral learning and hypercube recovery include the hardware simplicity offered by the use of conventional cameras, high temporal resolution if a video is used (e.g., slow-motion video recording on a smartphone), independence (no tradeoff) between the spatial and spectral resolutions, and abundant spectral information for a variety of machine-learning techniques. Locally sampled hyperspectral data can serve as prior information or physical constraints for incorporating domain-specific modeling into the learning algorithm, extracting critical features and parameters, and resulting in explainable and interpretable neural networks.

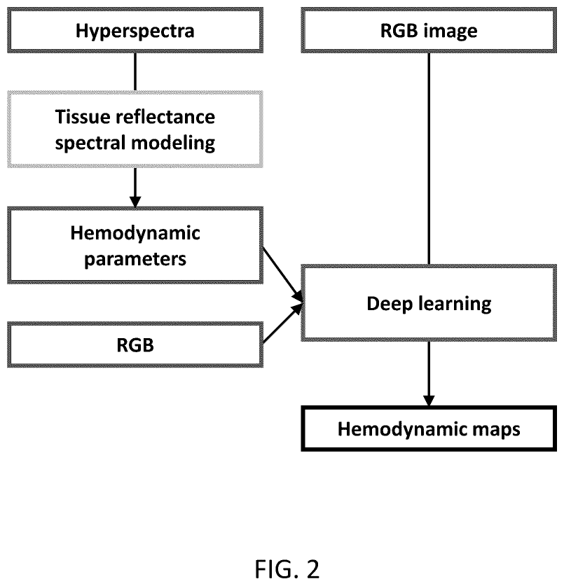

To instantaneously sample hyperspectral data in a small subarea, the trichromatic camera (e.g., smartphone camera) is combined with a line-scan spectrograph. Specifically, a dual-channel spectrograph with a photometric slit acquires an RGB image in the entire area and the hyperspectral data of a subarea (e.g., a central line) in a single-shot manner. The field-of-view may be as small as 2.5 mm×2 mm with a spatial resolution of 55 μm. The sampled hyperspectral data have a spectral range of λ=380-720 nm with a spectral resolution Δλ=0.5 nm. The dual-channel imaging setup provides sufficient training data (750-1500 data points) for the machine learning package (e.g., a neural network as further described in FIG. 2 and below). This dataset is randomly split into training (80%) and testing (20%) datasets for effectively training the hyperspectral learning algorithm to be applied to the entire area eventually (see FIG. 2). In addition, this imaging setup allows us to use a smartphone camera that can acquire videos at different frame rates. In particular, highly dynamic imaging is even possible with a high temporal resolution even up to 0.0005 sec for 1920 frames per second, using commercially available smartphone models.

Referring to FIG. 2, a block diagram is provided that outlines the steps of the present approach. Hyperspectral data from the slit is provided to a model of interest. For example, if hemodynamic parameters are of interest, the model may be a tissue reflectance spectral model. The output of the model will include parameters of interest. In the case of hemodynamics, the model's output includes hemodynamic parameters. These output parameters along with the RGB image from the same limited area corresponding to the slit are provided as input to a deep learning modeling package along with the RGB of the entire sample area. The deep learning package then provides the hemodynamic parameters for the entire sample viewing area in the form of a hemodynamic map. It should be appreciated by tailoring the tissue reflectance spectral model to other parameters of interest, the deep learning package may be configured to output selective maps of the entire sample image area.

The hyperspectral learning addresses an ill-posed problem, which is also known as spectral super-resolution and hyperspectral reconstruction. The mathematical relationship between the RGB and hyperspectral intensity is provided as:

$$\\ \begin{matrix}{x}_{3 \times 1} = {S}_{3 \times k} {y}_{k \times 1} + {e}_{3 \times 1} & ( 1 )\end{matrix}$$

where x denotes a 3×1 vector corresponding to three color values in the R, G, and B channels

$$\\ ( x = {[ R , G , B ]}^{T} ) ,$$

S represents a 3×k matrix of the RGB spectral response of the three-color sensor the spectral response functions in the R, G, and B channels of the smartphone camera (also known as the sensitivity function of the camera),
y is a k×1 vector that has the spectral intensity (y=[I(λ₁), I(λ₂), . . . , I(λₖ)]^{T}) where A is discretized in the visible range with a spectral interval of 1 nm, and
e symbolizes a 3×1 vector of the system noise. Thus, hyperspectral learning amounts to obtaining a pseudoinverse of S_{3×k}. Depending on the availability of training data and the desired spectral resolution, the machine-learning approach discussed herein is a deep learning approach using a neural network.

A key assumption for reliable hyperspectral learning is that a sampling distribution (i.e., RGB values of the sampled subarea) should follow the parent distribution (i.e., RGB values of the entire image area); i.e., the intensity distributions between the sampled subarea and the entire field-of-view of interest are about statistically the same. Specifically, the probability distribution of the R, G, and B values in the subarea needs to conform to those in the entire area in terms of variability and shape. In addition, to reliably predict unknown hyperspectral output responses from RGB values outside the subarea, the hyperspectral learning algorithm should be applied within the same (minimum and maximum) range of sampled RGB values used to train the algorithm. In a similar manner to nonparametric tests with non-Gaussian distributions, known to a person having ordinary skill in the art, quantile-quantile (Q-Q) plots can conveniently be used to assess if the two sets of data plausibly follow the same distribution within the same range. Validity of this assumption allows for interpolation from the subarea to the entire field which offers an important advantage over conventional snapshot hyperspectral imaging. If these assumptions are valid, then the hyperspectral learning is not limited by the intrinsic tradeoff between spatial and spectral resolutions.

This assumption can be tested and further optimized, by 1) changing the location of the subarea (i.e., position of the slit in FIG. 1), 2) dividing a frame into subframes, where the subframes each produce a more favorable assumption as compared to the entire frame, and 3) a combination of (1) and (2). These three methods can be combined with an optimization approach, e.g., least-squares, using a feedback signal in order to minimize error using a quantitative approach, e.g., Q-Q plots.

Spectrally informed learning allows for the incorporation of physical and biological understanding of domain knowledge into learning algorithms. Among the various snapshot imaging applications, we focus on extracting biological parameters or spectral signatures from a hypercube using the domain knowledge of tissue optics. In this perspective, light propagation in tissue can be explained by the theory of radiative transport and robust approximations (e.g., diffusion, Born, and empirical modeling). Specifically, taking advantage of tissue reflectance spectral modeling, we extract the key hemodynamic parameters: oxygenated hemoglobin (HbO₂), deoxygenated hemoglobin (Hb), and oxygen saturation (sPO₂), which are the fundamental determinants of oxygen transport to tissue associated with a variety of physiological changes, diseases, and disorders, as described below:

$$\\ \begin{matrix}sP O 2 = \frac{{Hb O}_{2}}{Total hemoglobin} = \frac{{Hb O}_{2}}{{Hb O}_{2} + Hb} & ( 2 )\end{matrix}$$

Notably, tissue optics serves as the cornerstone of biophotonics and biomedical optics to deepen our knowledge of light-tissue interactions and develop noninvasive optical diagnostic methods and devices. Typically, purely data-driven learning requires a large volume of training data and lacks explainable and interpretable learning. On the other hand, tissue optics modeling can offer insights into the black box nature of deep learning.

To demonstrate the versatility of hyperspectral learning and hypercube recovery, we formulate a deep learning approach (see FIG. 2), where hyperspectral data in a subarea is directly fed into the tissue reflectance spectral model to compute the hemodynamic parameters within the same subarea. The obtained dataset serves to train a deep neural network that computes the hemodynamic parameters with RGB values (tristimulus) as an input. Specifically, the deep neural network is directly trained by using the hemodynamic parameters extracted from the tissue reflectance spectral model fed with the sampled data, thereby reducing the computational load. In both cases, separate training and validation datasets are employed to strengthen the learning algorithm for training: 80% of the data points among the sampled data (i.e., 600 data points out of 750) are randomly selected as a training dataset and the remaining 20% (i.e., 150 data points) are blindly tested as a testing dataset.

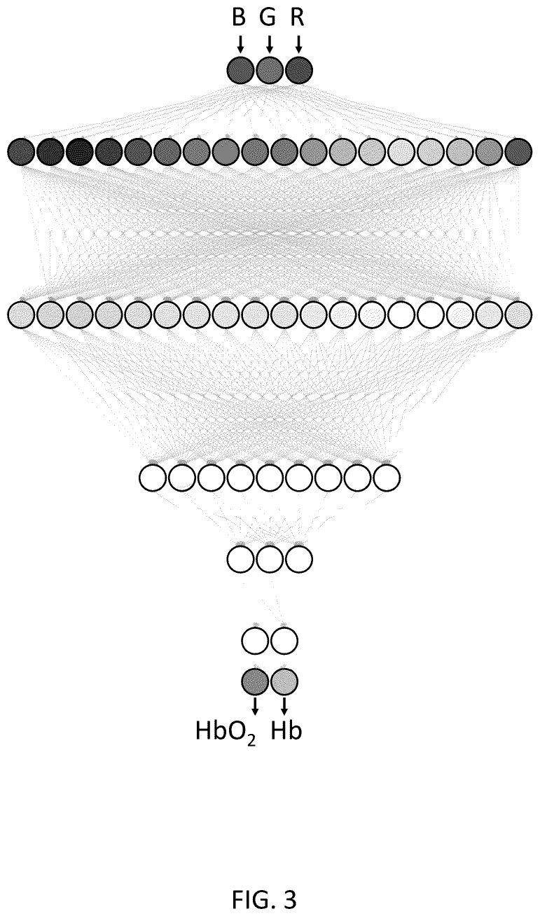

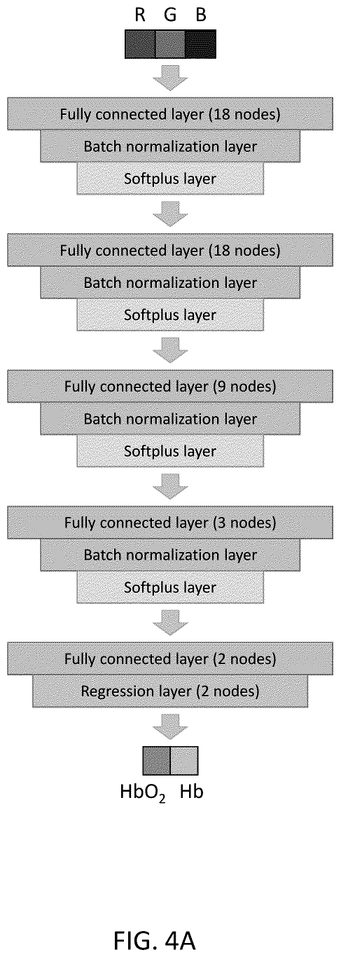

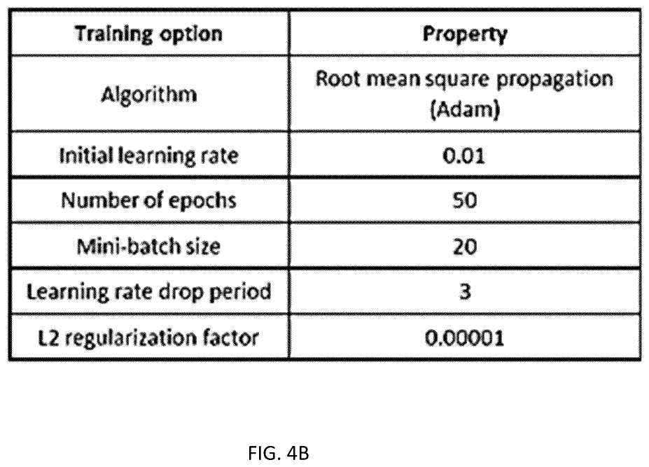

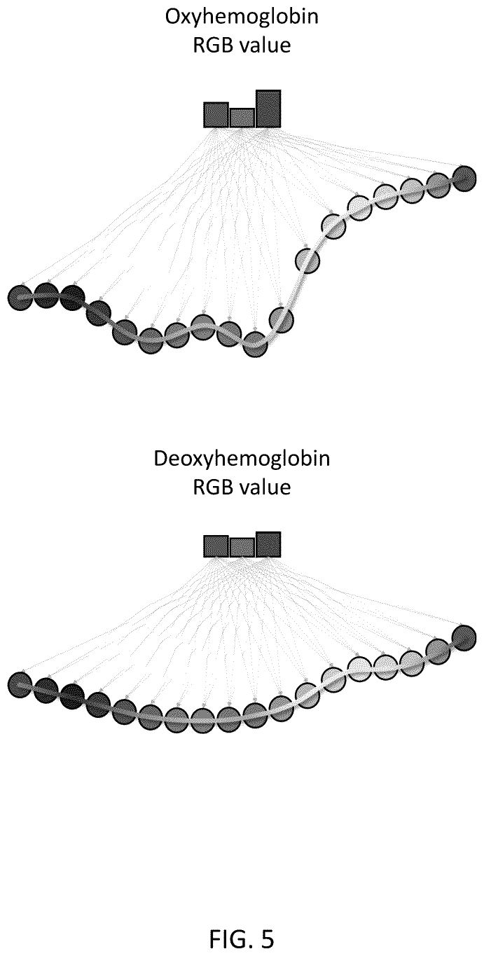

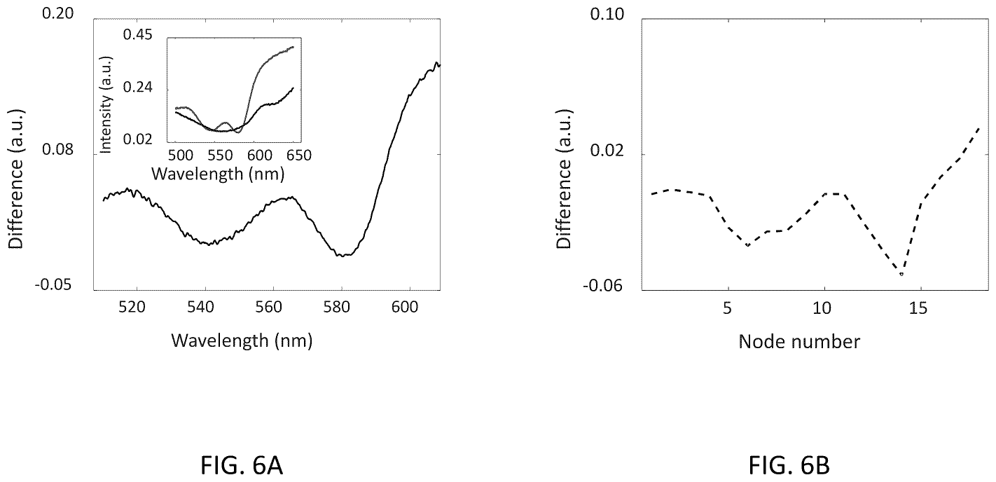

Importantly, deep learning informed by hyperspectral information is advantageous for designing explainable and interpretable neural networks. Among similar yet distinct terms, such as understandability and comprehensibility, spectrally informed deep learning enables transparency in the learning algorithm as it is understandable in a manner similar to statistical regression. A conceptual drawing of the neural network is shown in FIG. 3 (a conceptual illustration of the deep neural network that receives RGB values and returns key hemodynamic parameters (i.e., HbO₂ and Hb). This network is trained by the RGB values and hemodynamic parameters in a subarea; HbO₂ and Hb values are computed from the measured hyperspectral data in the subarea via the tissue reflectance spectral model), with more information provided in FIGS. 4A and 4B. Specifically, FIGS. 4A and 4B provide architecture diagram and key hyperparameters of the deep neural network that directly returns the hemodynamic parameters of Hb and HbO₂ from the RGB input values. Specifically, in FIG. 4A the network is informed by hyperspectral learning as well as tissue reflectance spectral modeling such that the output hemodynamic parameters are extracted from tissue reflectance spectral modeling. FIG. 4B provides training options and hyperparameters that are optimized to efficiently extract the hemodynamic parameters directly from the RGB values. The first hidden layer, which is one of the important hyperparameters for building a neural network, is fully connected with a relatively large number of nodes (e.g., 18 nodes), which transforms RGB values to a spectral intensity profile with a high spectral resolution. After the network is trained, each node in the first hidden layer possesses a distinct weight representing hyperspectral information, such that the RGB values of a certain hemodynamic parameter (e.g., sPO₂) generate the corresponding spectral feature, which is further propagated throughout the network as shown in FIG. 5 (the first hidden layer is fully connected to 18 nodes (or neurons)). FIG. 6A illustrates two representative cases of hyperspectral data measured from a tissue phantom by varying sPO₂ between HbO₂ and Hb. The output values at different nodes in the first hidden layer can be understood based on the spectral intensity differences as a function of λ (see FIGS. 6A and 6B) that a scientific spectrometer or spectrograph can quantify. FIG. 6A is a representative spectral intensity differences in hyperspectral data measured from a tissue phantom with oxygenated and deoxygenated hemoglobin. Inset: represents measured hyperspectral data when the sample is oxygenated (red curve) and deoxygenated (blue curve) (i.e., HbO₂ or Hb, respectively). FIG. 6B is a representative difference in the computed output values of the first hidden layer between two different RGB values of HbO₂ and Hb from the same tissue phantom in FIG. 6A. The order of the nodes is assigned such that the rank of the output differences is the same as that of the wavelengths in the spectral intensity differences. This direct spectral understanding should be differentiated from other conventional heatmaps or saliency maps employed for visualizing or explaining features extracted through typical convolutional neural networks. In particular, the differences in the computed output values of the first hidden layer between the two different RGB values of HbO₂ and Hb in a tissue phantom resemble the spectral intensity differences between HbO₂ and Hb measured from the same tissue phantom.

In the deep-learning framework (see FIG. 2), we design a fully connected deep neural network that takes RGB values as the input and returns the hemodynamic parameters as the output (see FIGS. 3 and 4A). As stated previously, the neural network incorporates the output parameters extracted from the tissue reflectance spectral modeling (see FIG. 5).

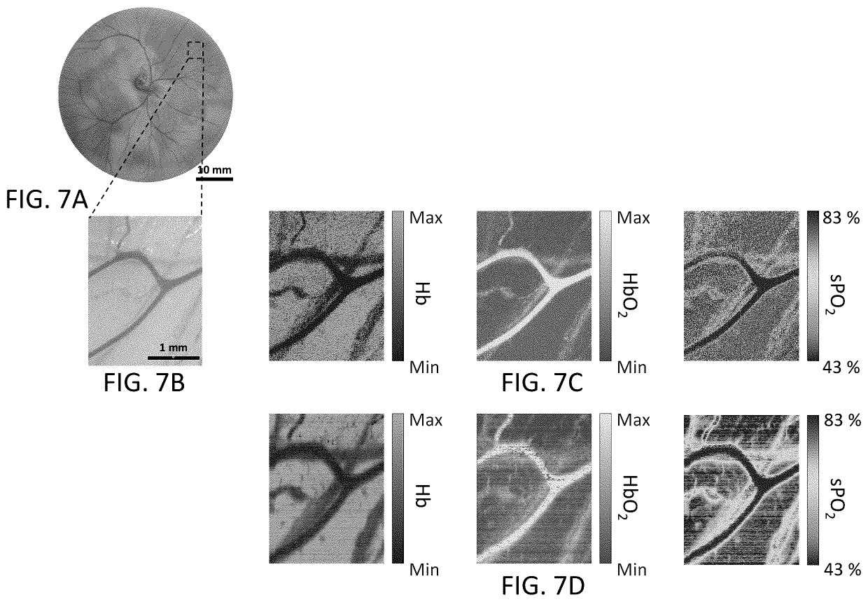

Referring to FIGS. 7A, 7B, 7C, and 7D, a chick embryo in a petri dish as an experimental vascular developmental model is shown in FIG. 7A. RGB image generated by the conventional pushbroom-type hyperspectral imaging system of the prior art is shown (FIG. 7B) and a ground truth for the hemodynamic map of Hb, HbO₂, and sPO₂ corresponding to a white leghorn chicken (Gallus domesticus, Hy-Line W-36) embryo on day 8 based on FIG. 7B is shown in FIG. 7C. The deep learning-based hemodynamic maps are shown in FIG. 7D. The hemodynamic maps (FIG. 7D) are directly generated using the deep neural network approach discussed herein that takes the RGB values as the input and returns HbO₂ and Hb values as the output. Reference hemodynamic maps that is generated by the conventional pushbroom-type hyperspectral imaging system used for validation is shown in FIG. 7C. The deep learning-neural network based hemodynamic map (FIG. 7D) is in excellent agreement with the ground-truth maps shown in FIG. 7C. Deep learning-based hemodynamic maps are further assessed using the structural similarity index to show that they are qualitatively identical to the reference hemodynamic maps as provided below. Interestingly, the conventional hemodynamic maps shown in FIG. 7C are noisier due to the motion artifact of the live sample, which results from the slow rate of data acquisition (data acquisition time=45 minutes) and the mechanical scanning, as shown by the horizontal lines in FIG. 7C.

TABLE 1
Structural similarity index values to compare learning-based hemodynamic
maps with a conventional pushbroom-type hyperspectral imaging system

| | | Hyperspectral learning − | | | Hyperspectral learning − | | |
|---|---|---|---|---|---|---|---|
| | | Deep learning | | | Statistical learning | | |
| | | Oxygenated | Deoxygenated | Oxygen | Oxygenated | Deoxygenated | Oxygen |
| | | hemoglobin | hemoglobin | saturation | hemoglobin | hemoglobin | saturation |
| Conventional | Oxygenated | 0.936 | | | 0.905 | | |
| scanning | hemoglobin | | | | | | |
| system | Deoxygenated | | 0.985 | | | 0.978 | |
| | hemoglobin | | | | | | |
| | Oxygen | | | 0.998 | | | 0.997 |
| | saturation | | | | | | |

The maximum value of structural similarity index is 1.0 if two images are identical.

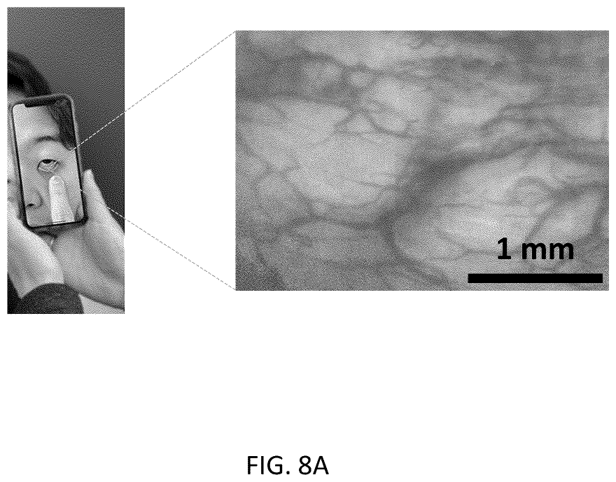

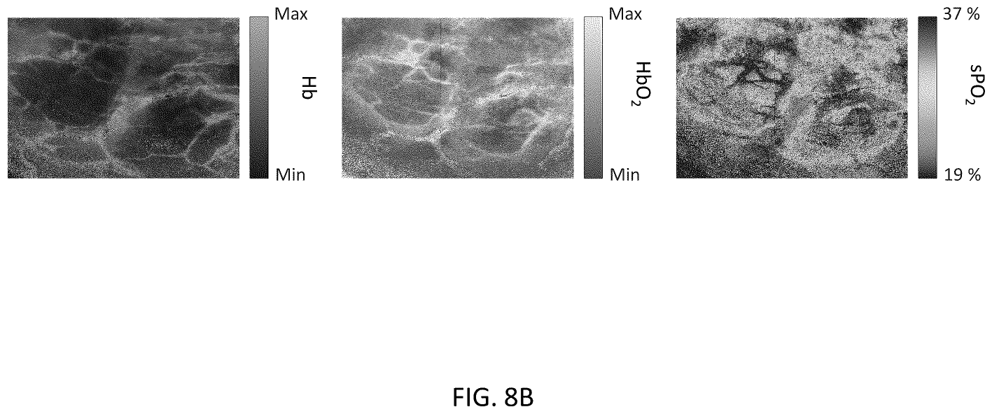

As a model system for peripheral microcirculation in humans, we visualize spatiotemporal hemodynamic changes in the microvessels of the inner eyelid (i.e., the palpebral conjunctiva), shown in FIG. 8A, which is a photograph of a healthy adult volunteer taking a picture with a smartphone while the inner eyelid is pulled down including an RGB image of the inner eyelid with high spatial resolution showing the field-of-view. Microvessels in the inner eyelid are clearly visible without the effects of skin pigments, which are easily accessible for imaging. The inner eyelid is an easily accessible and highly vascularized peripheral tissue site that receives blood from the ophthalmic artery. Thus, the inner eyelid serves as a feasible sensing site for various diseases and disorders. FIG. 8B shows the peripheral hemodynamic maps of Hb, HbO₂, and sPO₂ obtained during the resting state of a healthy adult volunteer. In FIG. 8B, the sPO₂ maps reveal spatially complex patterns of perfusion in the inner eyelid, which are not evident in the photo (i.e., the RGB image).

It should be appreciated that the RGB camera and the spectrograph camera can be replaced with a microscope adapted, wherein the microscope includes a fiber optics spectrometer which receives light via a beam-splitter thereby obtaining hyperspectral and RGB data in the subarea and an RGB image of the field of interest. An example of such a microscope set up is MICROSPECTROSCOPY made by HORIBA SCIENTIFIC.

Incorporation of a spectroscopic analysis into a learning algorithm is also within the scope of the present disclosure. Tissue optics has been the cornerstone of biophotonics and biomedical optics to deepen our knowledge about light-tissue interactions and develop noninvasive optical diagnostic methods and devices. Light propagation in tissue can be explained by the theory of radiative transport and robust approximations (e.g. diffusion, Born, Monte Carlo simulation, and empirical modeling). An understanding of tissue optics allows us to ensure that the resulting outputs and learning algorithms are explainable and interpretable, overcoming the black box nature of deep learning.

Those having ordinary skill in the art will recognize that numerous modifications can be made to the specific implementations described above. The implementations should not be limited to the particular limitations described. Other implementations may be possible.

1. A method of generating an image or video of a field of interest of a sample, comprising:
Obtaining: i) a first Red-Green-Blue (RGB) image from about a field of interest of a sample, and ii) hyperspectral data from a subarea of the field of interest;
extracting an RGB image of the subarea from the first RGB image of the field of interest;
applying the hyperspectral data of the subarea to conduct a spectroscopic analysis of a sample, thereby generating spectral parameters for the subarea;
inputting i) the generated spectral parameters, and ii) the extracted RGB image of the subarea, collectively as training input data to a deep learning model;
training the deep learning model with the training input data thus generating a trained deep learning model;
obtaining a second RGB image about the field of interest including areas outside of the subarea;
inputting the second RGB image of the field of interest to the trained deep learning model; and
outputting from the trained deep learning model a spectral map for the field of interest.

2. The method of claim 1, wherein the deep learning model is a neural network.

3. The method of claim 1, wherein the first and second RGB images of the field of interest are obtained as a photograph.

4. The method of claim 1, wherein the first and second RGB images of the field of interest are obtained from a video frame.

5. The method of claim 4, wherein the video has a frame rate of between about 960 frames per second and about 1920 frames per second.

6. The method of claim 1, wherein the first and second RGB images are obtained from a trichromatic camera.

7. The method of claim 1, wherein the hyperspectral data is obtained from an imaging spectrograph.

8. The method of claim 1, wherein the hyperspectral data is obtained from a spectrometer.

9. The method of claim 6, wherein the trichromatic camera is a smartphone camera.

10. The method of claim 6, wherein light from the sample is provided to the trichromatic camera via a mirror and a first plurality of lenses.

11. The method of claim 7, wherein the mirror includes a photometric slit adapted to provide light from the subarea.

12. The method of claim 7, wherein light exiting the photometric slit is diffracted by a diffraction grating and supplied to the imaging spectrograph via a second plurality of lenses.

13. The method of claim 7, position of the photometric slit on the mirror is selectable.

14. The method of claim 10, wherein the first and second RGB images from the field of interest are dividable into two or more subareas and for each said subarea, position of the photometric slit on the mirror is selectable.

15. The method of claim 1, wherein the first and second RGB images are obtained from a microscope combined with a fiber optics spectrometer via a beam-splitter thereby enabling obtaining hyperspectral and RGB data in the subarea and an RGB image of the field of interest.

16. The method of claim 1, wherein the spectroscopic analysis of the sample is based on a sample optics domain knowledge model to generate the spectral parameters from the hyperspectral data.

17. The method of claim 14, wherein the sample optics domain knowledge model is based on one or more of theory of radiative transport, robust approximations, and Monte Carlo simulations.

18. The method of claim 15, wherein the robust approximations are based on one or more of diffusion, Born, and empirical modeling.

19. The method of claim 1, wherein the spectral parameters include hemodynamic parameters.

20. The method of claim 1, wherein the first RGB image is same as the second RGB image.
